SEQUENCE LISTING (5 sequences)

SEQ ID NO: 1 - DNA, 20 bp, Artificial Sequence
  Description of Artificial Sequence Synthetic primer
gggaccaccgacccaccacc

SEQ ID NO: 2 - DNA, 20 bp, Artificial Sequence
  Description of Artificial Sequence Synthetic primer
gatcctcatcctgtctcttg

SEQ ID NO: 3 - DNA, 18 bp, Artificial Sequence
  Description of Artificial Sequence Synthetic primer
atcctcgaagatctaggg

SEQ ID NO: 4 - DNA, 31 bp, Artificial Sequence
  Description of Artificial Sequence Synthetic primer
caatatcgattactagcgaactaatctagga

SEQ ID NO: 5 - DNA, 44 bp, Artificial Sequence
  Description of Artificial Sequence Synthetic primer
atatcgattactagcgaactaatctacgacccccgtaaaggtgt

(19) United States
(12) Patent Application Publication
(10) Pub. No.: US 2008/0118956 A1
(43) Pub. Date: May 22, 2008

(54) Viral Particles Containing An Alphavirus-Derived Vector And Method For Preparing Said Viral Particle

(76) Inventor: Jean-Christophe Pages, Lussault Sur Loire (FR)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 10/581,401

(22) PCT Filed: Nov. 30, 2004
(86) PCT No.: PCT/FR04/50631

§ 371 Date: Sep. 25, 2007

(30) Foreign Application Priority Data
Dec. 2, 2003 (FR) 0350951

(51) Int. Cl. C12N 15/64; C12N 15/00

(52) U.S. Cl. 435/91.42; 435/320.1

(57) ABSTRACT

Viral particle consisting of structural elements not derived from an alphavirus and containing an alphavirus-derived vector made replication-defective by deletion, or replacement with at least one transgene, of the structural genes, characterized in that the structural elements of said particle are not encoded by the genome of the alphavirus-derived vector. Method for producing said particle, which consists in expressing in trans, in a cell line, the genes encoding the structural elements not derived from the alphavirus and the alphavirus-derived vector, and then in recovering the viral particles present in the cell culture supernatant.

The invention relates to novel viral particles containing a vector derived from an alphavirus made defective with respect to autonomous propagation and therefore with respect to replication. It also relates to the method for preparing said particles.

In the subsequent description, the invention is more particularly illustrated in relation to the Semliki forest virus (SFV) that falls within the category of the alphaviruses. Of course, this particular example in no way limits the scope of the invention and all alphaviruses can be envisaged, for instance the Sindbis virus.

The alphavirus genome is in the form of a single-stranded RNA with positive polarity comprising two open reading frames, respectively a first frame encoding the proteins with enzymatic function and a second frame encoding the structural proteins. Replication takes place in the cytoplasm of the cell. In the first step of the infectious cycle, the 5′ end of the genomic RNA is translated into a polyprotein (nsP 1-4) with RNA polymerase activity that produces a negative strand complementary to the genomic RNA. In a second step, the negative strand is used as a template for the production of two RNAs, respectively:
a positive genomic RNA corresponding to the genome of the secondary viruses producing, by translation, other nsp proteins and acting as a genome for the virus,
a subgenomic RNA encoding the structural proteins of the virus forming the infectious particles.

More specifically, the subgenomic RNA is transcribed from the p26S promoter present at the 3′ end of the RNA sequence encoding the nsp4 protein. The positive genomic RNA/subgenomic RNA ratio is regulated by proteolytic autocleavage of the polyprotein to nsp 1, nsp 2, nsp 3 and nsp 4. In practice, the viral gene expression takes place in two phases. In a first phase, there is main synthesis of positive genomic strands and of negative strands. During the second phase, the synthesis of subgenomic RNA is virtually exclusive, thus resulting in the production of a very large amount of structural proteins.

Knowledge of the method of replication of alpha-viridae and the simplicity of their genome had led to the emergence of gene transfer systems using these viruses, the latter making it possible to obtain strong expression of the transgene in the target cell.

One of the essential conditions for it to be possible for an alphavirus-derived vector to be used in gene therapy is that it exhibit no ability to replicate. Several solutions have been proposed for rendering the Semliki virus replication-defective.

The first solution consists in deleting the structural genes of the Semliki RNA to the benefit of the transgene, the transgene being placed under the control of the p26S promoter. Such a vector can be transferred to cells in the form of RNA or in the form of DNA. However, this solution is not very advantageous for in vivo applications, insofar as a poor transfer efficiency is observed with these genetic elements used in the absence of particles.

Another solution consists in infecting the target cells with a Semliki vector, not in the form of DNA or of RNA alone, but in the form of recombinant viral particles. To do this, a cell line is transfected with at least two plasmids, respectively a plasmid carrying the Semliki vector RNA devoid of structural genes and a second plasmid carrying the Semliki structural genes under the control of the p26S promoter. Viral particles are formed in the cell, which particles encapsidate only the defective RNA, i.e. the Semliki RNA carrying the transgene, since only this RNA also carries an encapsidation sequence contained in the sequence of nsP2. Even though, in theory, this process generates no replicative particle, recombination events remain frequent, in particular because of the overlap between the complementation sequences and the sequences of the recombinant virus, and because of the abundance of viral RNAs in the cytoplasm of the producer cells.

The Rolls documents (1, 2) describe an SFV vector in which the genome has been modified by replacement of the structural genes with the gene encoding the VSV-G envelope, optionally combined with a transgene. Infectious particles thus obtained therefore consist of a VSV-G envelope and contain an alphavirus-derived vector. However, the system described is particularly dangerous because of its ability to replicate autonomously. An equivalent system is described in document WO 03/072771.

The document Lebedeva et al. (3) describes the coelectroporation of BHK cells with:
a vector providing the replication functions (Srepβgal: gene encoding the Semliki (SFV) vector replicase and β-galactosidase as transgene),
a vector encoding the structural genes: ScapSenv encoding the capsid and the envelope of SFV as a control, or a derived vector in which the env gene of SFV is replaced with env sequences of the MLV (Moloney murine leukemia) retrovirus,
   and the analysis of the viral particles thus produced. However, in this system, the mobilization of the SFV vector transporting the transgene, in this instance that of β-galactosidase, is under the control of the encapsidation of this recombinant genomic RNA by the SFV capsid protein.

In other words, the problem that the invention proposes to solve is that of improving the method of mobilization of alphavirus-derived vectors, in particular of the Semliki forest virus (SFV), so as to prevent any risk of recombination within the producer lines that may generate replicative particles.

Another problem that the invention proposes to solve is that of preparing viral particles containing an alphavirus-derived vector, the tropism of which is not limited to the target cells of the wild-type viruses.

The Applicant has succeeded in producing viral particles that correspond simultaneously to the two objectives above, by expressing, in trans, in a cell line, the genes encoding structural elements not derived from the alphavirus, and the alphavirus-derived vector made replication-defective.

According to a first embodiment, the genes encoding structural elements not derived from the alphavirus correspond to only the ENV gene of the vesicular stomatitis virus, encoding the VSV-G envelope protein.

The use of a VSV-G envelope has several advantages. First of all, the envelope protein of the vesicular stomatitis virus allows a method of cell entry by endocytosis that can be superposed on that of alphaviruses. In addition, VSV-G is a very stable protein that can be concentrated by ultracentrifugation and makes it possible to envisage parenteral administrations. Moreover, this protein confers a very broad tropism on the particles that contain it, thus enlarging the field of use of the viral particles of the invention to organisms as different as drosophila and mammals.

According to this first embodiment, the expression in trans is obtained by cotransfection advantageously carried out in two distinct steps, respectively the transfection of the line with the plasmid expressing the VSV-G envelope gene, and then a second transfection with the alphavirus-derived vector. In practice, the cotransfection is performed on 293T cells.

In a second embodiment, the genes encoding the structural elements not derived from the alphavirus correspond to the genes encoding the structural proteins of a retrovirus.

In this case, the expression in trans is obtained by transfection of an encapsidation cell line, that produces replication-defective retroviruses, with the alphavirus-derived vector. This type of vector is well known to those skilled in the art, for example the Phoenix® system (http://www.stanford.edu/group/nolan/retroviral systems/phx.html). Encapsidation lines that use structural genes of MLV (murine leukemia virus) can in particular be used.

In a known manner, these lines are obtained by stable transfection of a first plasmid expressing the GAG-POL genes and of a second plasmid expressing an ENV gene of a retrovirus or of another enveloped virus (4).

However, it is also possible to envisage preparing the viral particles by triple transfection of a cell line, for example 293T cells, by introduction of a first viral element expressing the retroviral GAG and POL genes, of a second viral element expressing the retroviral ENV gene and of the alphavirus-derived vector.

It is possible to even further accentuate the defective nature of the transcomplementing retroviral sequences by mutation, in particular deletion of the nucleotide sequences of the POL gene encoding the integrase (IN) and the reverse transcriptase (RT).

In the two embodiments of the invention as described above, the alphavirus-derived vector is made replication-defective. This property is in practice obtained by deleting the structural genes or substituting them in favour of the transgene(s) of interest in the genome of the vector.

According to another characteristic, the genome of the alphavirus-derived vector contains a signal for encapsidation by the viral particle, called psi sequence.

According to a first embodiment, the psi sequence corresponds to the extended packaging sequence of MLV vectors, obtained by amplification, according to the PCR (polymerase chain reaction) method, of the PLNCX vector (Clontech®) using the primers:

5′ primer:
LNCX Psi 2a: 5′-GGGACCACCGACCCACCACC-3′
and
3′ primer:
LNCX Psi 2b: 5′-GATCCTCATCCTGTCTCTTG-3′.

Advantageously, the psi sequence is small in size and corresponds to the minimal sequence. This modification is advantageous insofar as the psi sequence can function as an anchoring point for ribosomal entry (IRES). The IRES function thus makes it possible to eliminate the SFV p26S promoter, such that the translation of the transgene is obtained from the genomic RNA.

Paradoxically, the Applicant has also demonstrated that the presence of a retroviral encapsidation signal is not absolutely necessary. In fact, the amount of recombinant RNAs of the Semliki vector, found in the cytoplasm of the transfected cells, is such that said RNAs are preferentially encapsidated in the retroviral particles. This phenomenon is accentuated by the quenching of the cellular genes, induced by the expression of the non-structural proteins of the Semliki virus. The subcellular localization of the SFV virus replication complexes could also play an important role (5). Consequently, and in a preferred embodiment, the genome of the vector is devoid of psi sequence.

The applicant has also shown that it is possible to mobilize a vector as described above, containing a retroviral encapsidation sequence, by means of retroviral particles produced using a trans-complementation system based on vectors derived from the Semliki forest virus (11). In this system, it is shown that titres of the order of 10⁶ particles per millilitre can be obtained. It is also demonstrated that the presence of a retroviral encapsidation signal improves the particle titre by approximately one log. These particles are used effectively for transducing cells expressing the amphotropic virus receptor (Pit 2) corresponding to the retroviral envelope used. This observation has a direct consequence on the biosafety of the retroviral particles produced by the method of Li and Garoff (11). In this context, it is demonstrated that the particles produced according to the method of Li and Garoff contain, at a titre close to 10⁶ particles/ml, recombinant SFV vector genomes expressing the retroviral GAG/POL or ENV sequences.

The Applicant has, moreover, noted that the method of transfection generally used for recombinant RNAs of Semliki vectors, namely electroporation, results in substantial cell suffering. Thus, and in order to allow transfection of the producer cells by methods that are milder than electroporation, the alphavirus-derived vector was modified so as to be expressed from a eukaryotic promoter, for example a CMV promoter positioned 5′ of the vector sequence.

Finally, the p26S promoter of the alphavirus vector is advantageously mutated. The SFV 26Sm2 vector, and to a lesser extent the SFV 26Sm1 vector, no longer expresses any detectable subgenomic RNA that may decrease genomic RNA encapsidation by competition.

Thus, a particle according to the invention corresponds to a viral particle consisting of structural elements not derived from an alphavirus and containing an alphavirus-derived vector made replication-defective by deletion, or replacement with at least one transgene, of the structural genes, the structural elements of said particle not being encoded by the genome of the alphavirus-derived vector.

Moreover, the invention relates to the use of the viral particles according to the invention, for infecting cells in vitro. The Applicant has shown that the particles thus produced can infect a large variety of eukaryotic cells, both human and nonhuman.

The invention also relates to a pharmaceutical composition comprising the viral particles of the invention.

Similarly, it relates to the use of the viral particles, for preparing a medicinal product for use in the treatment of cancer.

The invention and the advantages that ensue therefrom will emerge clearly from the following examples.

FIG. 1 is a diagrammatic representation of the structure of the Semliki forest virus (SFV)-derived vector.

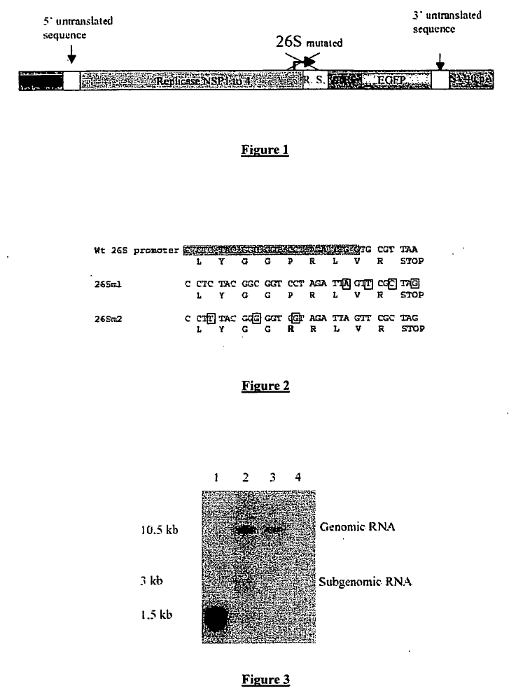

FIG. 2 shows the mutations effected in the p26S promoter. The mutations introduced into the mutants p26Sm1 and p26Sm2, relative to the wild-type sequence (Wt), are boxed in. The amino acid in bold indicates a change in the coding sequence.

FIG. 3 is the result of a Northern blot performed using producer cells, expressing modified SFV vectors (1:pEGFPC1; 2:p26Sm1; 3:p26Sm2; 4:SFV without transgene), with a GFP probe from pEGFPC1.

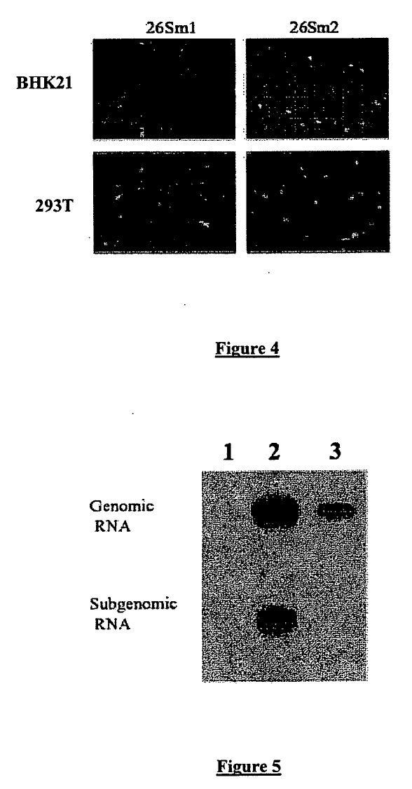

FIG. 4 shows the ability of the 293T and BHK 21 cells to express the SFV-derived vectors (p26Sm1 and p26Sm2) mobilized by the VSV-G pseudoparticles.

FIG. 5 is a result of a Northern blot performed using cells infected with the supernatant of 293T cells transfected with the pMDG plasmid and modified SFV vectors (1:pEGFPC1; 2:p26Sm1; 3:p26Sm2), with a GFP probe from pEGFPC1.

### EXAMPLE 1

### Production of Viral Particles from Cell Lines Expressing the VSV-G Envelope

### 1/METHODS

1/Cell Lines and Cultures
293T/17: primary human embryonic kidney line (ATCC CRL-11268),
Hela: human cell line (ATCC CCL-2),
QM7: quail muscle line (ATCC CRL-1962),
LMH: chicken liver line (ATCC CRL-2117).

The four cell lines above are cultured in DMEM (Invitrogen) containing 10% of foetal calf serum (FCS) (Biowest).
HepG2: human hepatoma line cultured in EM containing 10% of FCS (ATCC HB-8065),
BHK21: baby hamster kidney line cultured in GMEM containing 5% of FCS and 8% of liquid tryptose phosphate solution (ATCC CCL-10),
CESC: chick embryo obtained and cultured according to reference 26,
High Five cell cultured at 27° C. in Grace's insect medium (cat no. B85502 Invitrogen) containing 10% of FCS,
Sp2/O: murine lymphoplastoid line cultured in RPMI 1640 containing 10% of FCS (ATCC CRL1581).

### 2/Construction of the SFV Vector

The structure of the SFV vector is represented in FIG. 1.

a/26Sm1 vector

The 26S internal promoter of SFV is mutated by PCR using the vector pSFV1 (Invitrogen), which is devoid of structural genes and used as a template in the presence of two primers, respectively:
a primer 26Sm1F containing the Bgl II restriction site which appears in bold in the following sequence: 5′-ATCCTCGAAGATCTAGGG-3′,
a second mutated primer 26Sm1R containing the Cla I restriction site which appears in bold in the following sequence:

5′-CAATATCGATTACTAGCGAACTAATCTAGGA-3′.

Silent mutations are then introduced into the p26S promoter so as to give the p26Sm1 promoter as represented in FIG. 2. The product thus amplified is then cloned into a plasmid pIRES2-EGFP (Invitrogen) (FIG. 1). A retroviral sequence, denoted RS, derived from an MLV virus, is then inserted between the mutated 26S promoter and the IRES sequence. The fragments containing the mutated 26S sequence, the retroviral MLV sequence and the EGFP gene are then excised with Bgl II and Hpa I, and then cloned into the vector pSFVl between the Bgl II and Sma I restriction sites. The 10.5 kbp fragment containing the modified SFV replicon is finally cloned between the CMV IE promoter and the SV40 polyadenylation signal pA in a vector pIRES2-EGFP in which the IRES GFP sequence has been deleted.

b/Vector SFV26Sm2

The internal promoter is mutated by PCR using the plasmid SFV1 used as a template in the presence of two primers, respectively, a first primer 26Sm1F and a second primer 26Sm2R containing a restriction site that appears in bold in the following sequence:

5′-ATATCGATTACTAGCGAACTAATCTACGACCCCCGTAAAGGTG
T-3′.

The primer 26Sm2R brings about the modifications of the p26S promoter as illustrated in FIG. 2. The amplified product is then digested with Bgl II and Cla I and ligated into the vector 26Sm1, also digested with Bgl II and Cla I so as to delete the corresponding fragment.

### 3/Transfection of the 293T Cell Line with the SFV vectors 26Sm1 or 26Sm2 and the Plasmid pMDG and Collection of the Viral Particles

A transient transfection of 293T cells by means of a calcium/phosphate transfection kit (Invitrogen) is carried out. The 293T cells are seeded 8×10⁵ cells per well on 6-well plates and incubated at 37° C. overnight, before transfection. The transfection is carried out in two steps. On the first day, the 293T cells are transfected with 5 μg of a plasmid PMDG containing the gene encoding the VSV-G envelope, under the influence of a CMV IE promoter (6). In a second step, on the second day, the cells are transfected with 5 μg of the SFV vectors 26Sm1 or 26Sm2. The second transfection medium is left in contact with the cells for between 13 and 17 hours. On day no. 3, the medium is removed and replaced with fresh medium, allowing the release of the infecting particles. The culture medium containing the viral particles is collected 5 to 6 hours later.

### 4/Transfection of the BHK21 Cell Line with the SFV Vectors 26Sm1 or 26Sm2 and the Vectors SFV GAGPOL and SFV ENV and Collection of the Viral Particles

BHK21 cells are electroporated at 5×10⁶/ml (i.e. 4×10⁶ cells), at a voltage of 350 V, and a capacitance of 750 μF. The RNAs used for the electroporation, corresponding to the various vectors (26Sm1 or m2, SFV GAGPOL and SFV ENV), are transcribed using 1.5 μg of linearized DNA by means of an Invitrogen Sp6 polymerase kit. For the electroporation, 22 μl of the transcription product are electroporated. The recombinant particles are harvested 14 to 16 hours later. The supernatants are filtered and deposited onto the target cells in the presence of 2 μg/ml of polybrene.

### 5/Infection of the Cell Lines with the Viral Particles

The supernatant of the transfected 293T cell lines is collected and then filtered through a 0.45 μm filter (HA Millex®, Millipore), and then incubated with various cell lines, in the presence of a fresh medium containing polybrene, used at 5 μg per ml (Sigma). GFP expression in the infected cells is verified by means of an Olympus IX50 fluorescence microscope. The transfection is quantified by means of a Becton Dickinson FACScalibur® flow cytometer. For the control tests, the supernatants are used in various reagents:
10 μg per ml of RNAse A (Sigma),
1 μg per millilitre of actinomycin D (Sigma),
100 units per millilitre of DNAse I (Invitrogen),
1 mg per millilitre of geneticin (Sigma), and
3 μg per millilitre of puromycin (Cayla).

### 6/Concentration of Viral Particles

The supernatant of the transfected 293 cells is centrifuged at 150,000 g in an SW41 rotor for one hour at 4° C. The concentrated viruses are resuspended in 300 μl of PBS and 25 μl of the solution are used to infect 5×10⁵ cells (293T, BHK-21, Hela, HepG2, Sp2/O, LMH, QM7).

### 7/Northern Blotting

The RNA of the 10⁶ transfected or infected cells is extracted by means of a total RNA isolation system (Promega®). The RNA of untransfected 293T cells is extracted as a control. 2 μg of each RNA are subjected to electrophoresis on a denaturing formaldehyde gel and the RNA is transferred onto a positively charged nylon membrane (Hybond-XL; Amersham). The Northern blotting hybridization is carried out according to standard procedures. The probes correspond to a 790 bp Age I-BamH I GFP fragment of the plasmid pEGFPC1 (Clontech), the fragment being labeled (Rediprime® II DNA labeling system; Amersham) and column-purified (ProbeQuant® G-50 Micro Columns; Amersham) before use.

### II/RESULTS

### 1/Vector Functionality

The vectors SFV 26Sm1 and 26Sm2 correspond to SFV vectors in which the 26S promoter has been mutated with the aim of preventing any possible competition between the packaging of the SFV genomic RNA and the subgenomic RNA produced by transcription under the influence of the 26S promoter. The functionality of the two vectors was verified by transfection of 293T cells. The strong expression of GFP observed suggests that the transcription and the translation of the modified SFV vector are correct. This first result has been confirmed by Northern blotting analysis on the RNA extracted from 293 cells transfected with the SFV 26Sm1 vector.

As shown in FIG. 3, lane 2, the GFP probe reveals the existence of two bands corresponding to the genomic RNA and the subgenomic RNA, the latter suggesting that the 26S promoter is still functional.

The same test is carried out on the second vector, SFV 26Sm2, comprising additional mutations. The detection of GFP and the Northern blotting analysis confirm that the mutations introduced into the 26Sm2 promoter inhibit the production, by transcription, of the subgenomic RNA (see FIG. 3, lane 3).

### 2/Production of Viral Particles

293T cells are cotransfected with the plasmid pMDG and then the vector SFV 26Sm1 or SFV 26Sm2 as indicated above. The supernatant of the transfected cells is transferred onto fresh 293T cells or BHK 21 cells. The strong and rapid expression of GFP obtained shows that it is possible to mobilize SFV vectors by means of cells expressing the VSV-G envelope (FIG. 4).

### 3/Ability of the Viral Particles Obtained to Infect the BHK21, 293T and QM7 Cell Lines

The results are given in the table below.

The viral titres are detected 24 hours after infection, by FACS analysis. The percentage of cells expressing GFP, relative to the number of cells on the day of infection, makes it possible to calculate a recombinant particle titre (IP/ml).

TABLE 1

| | | | Concentrated |
|---|---|---|---|
| | Viral titre | Viral titre | 26Sm2 particles |
| Cell lines | 26Sm1 (IP/ml) | 26Sm2 (IP/ml) | (IP/ml) |
| BHK21 | 1.1 × 10⁶ | 0.9 × 10⁶ | 10⁷ |
| 293T | 1.5 × 10⁵ | 1.5 × 10⁵ | 5 × 10⁶ |
| QM7 | 3 × 10⁵ | 3 × 10⁵ | ND |

As this table shows, the highest titre is obtained with the BHK21 cells compared with the 293T and QM7 cells.

### 4/The Expression of GFP Involving the Target Cells is Due to a Real Transduction by the SFV Viral Particles

In order to be sure that the expression of GFP is due to the expression of the SFV vectors and not to the mobilization of plasmids derived from the initial transfection or a pseudotransduction of free GFP, the following controls are carried out.

First of all, the SFV RNA is detected by Northern blotting using RNA extracted from the infected cells (see FIG. 5). As for the producer cells, both genomic RNA and subgenomic RNA are observed in the cells infected with the SFV 26Sm1 vector. On the other hand, in the cells infected with the SFV 26Sm2 vector, only the genomic RNA is detected. The intensity of the signal suggests a strong replication of the SFV vectors. However, in order to be sure that the strong expression of GFP in the target cells indeed corresponds to the mobilization of SFV RNA, and that the plasmids have indeed been transferred into the target cells, in this instance 293T cells, DNase I at high concentration (1000 IU/ml) is added to the transduction supernatant. The SFV viral particle titres are similar to the titres obtained in the absence of DNase I, which suggests a transduction rather than a second transfection. However, such a result could be obtained should the plasmid be encapsulated in the transfected cells after its entry, and subsequently delivered into the transduced cell. In order to check for this possible phenomenon, the target cells are pretreated with actinomycin D at one microgram per millilitre, and then incubated with the infectious supernatant. Actinomycin D inhibits the expression of genes controlled by POL II RNA, like the genome of the SFV vector in the plasmid pSFV26Sm1 or m2, but has no action on SFV replicase. Similar expression of GFP is observed in the presence or in the absence of actinomycin D, which confirms that it is clearly an RNA that is transferred (see table 2).

The question of whether the GFP expression is indeed due to the expression of the SFV vectors or to a pseudotransduction in the target cells is then verified. This is because some publications (7) have shown that GFP can be transferred passively via retroviral particles independently of any expression. In order to be sure that this is not the case, the target cells are pretreated with two translation inhibitors, respectively geneticin and puromycin. After treatment, the target cells show barely detectable GFP expression, which shows that the GFP observed results from a translation and not from a passive transfer (table 2). In addition, the cotransfection of a plasmid pEGFPC1 strongly expressing GFP, with a plasmid encoding VSV-G, does not result in any pseudotransduction of the GFP. Similarly, the supernatants originating from the cells transfected with SFV vectors alone do not induce GFP expression, which proves that VSV-G must be present in order to promote the formation of the pseudoparticles. In order to confirm that the SFV RNA is protected in the VSV-G vesicles, the supernatants are treated with RNase A, before transduction. It appears that the RNase A treatment has no effect on the infectious titres, confirming that the SFV RNA is really protected (table 2). In view of all these results, it is deduced that the GFP expression in the target cells is due to a real transduction by the SFV viral particles.

TABLE 2

| | Not treated | Actinomycin D | Dnase I | Rnase I | Geneticin | Puromycin |
|---|---|---|---|---|---|---|
| Viral titre | 5 × 10⁵ | 7.5 × 10⁵ | 7 × 10⁵ | N.D. | N.D. | N.D. |
| BHK21 (IP/ml) | | | | | | |
| Viral titre | 3 × 10⁴ | N.D. | 3 × 10⁴ | 2 × 10⁴ | 9 × 10³ | 0 |
| 293T (IP/ml) | | | | | | |

### EXAMPLE 2

### I/METHODS

### 1/Constructs:

The constructs described in example 1 were used.

Two other derived constructs, exhibiting a substitution of the CMV promoter with the SP6 prokaryotic promoter, were also used:
The first construct, spSFV26Sm1 , is directly derived from SFV26Sm1.
The second construct, spSFV26Sm1Ψ, is obtained by Bgl II-Sma I digestion of a plasmid pSFV1 (Invitrogen®), into which is cloned a Bgl II-Hpa I fragment of the pIRES2 GFP plasmid (Clontech®), modified by the introduction of a PCR fragment containing the 3′ end of the nsp4 gene and generated using the primers 26Sm1F and 26Sm1R (cf. example 1, section 2a).

These constructs are transcribed in vitro, and the RNAs are then introduced by electroporation into the producer cells. The in vitro transcription is carried out after linearization of the plasmids by BstB I cleavage. The transcription is carried out in the presence of a cap analogue (Invitrogen®), of SP6 polymerase (Invitrogen®) and of ribonucleotides (Promega®).

### 2/Cells:

The 293-cell-derived Phoenix® recombinant retrovirus-producing cells (http://www.stanford.edu/group/nolan/retroviral systems/phx.html) are cultured in DMEM medium (GIBCO) in the presence of decomplemented foetal calf serum (Abcys).

The producer cells are transfected with the plasmids SFV26Sm1 or 26Sm2, at 4 μg of DNA per 5×10⁵ cells, in a well of a six-well plate. The transfection is carried out using calcium phosphate (calcium phosphate transfection kit, Invitrogen®).

For the two constructs expressing the SFV vectors in the form of RNA, the transfection is carried out by electroporation: 40 μl of replicons produced in vitro are placed in the presence of 40×10⁵ cells and electroporated using the EasyjecT Plus system (Equibio®).

20 hours after transfection, whatever the transfection method used, the medium is changed. 16 hours after this change, the medium is harvested in order to carry out the infections. During the harvest, the medium is filtered using 0.45 μm filters (Millipore®).

### 3/Infections:

The filtered supernatants are used to infect 293T cells, placed in culture in 12-well plates. The infection is carried out in the presence of a polycation necessary for the virus/cell interactions, polybrene (Sigma®) at 5 μg/ml. On the day of infection, a well of 293T target cells is trypsinized for counting.

24 hours after infection, the cells are trypsinized so as to be analysed by flow cytometry (FACScalibur, Becton-Dickinson®). The percentage of cells expressing GFP, relative to the number of cells on the day of infection, makes it possible to calculate a recombinant particle titre (IP/ml) (table 3).

### 4/Controls:

Controls, identical to those performed in example 1, were carried out:
10 μg per ml of RNAse A (Sigma®),
1 μg per millilitre of actinomycin D (Sigma®),
100 units per millilitre of DNAse I (Invitrogen®),
1 mg per millilitre of geneticin (Sigma®).

### II/RESULTS

### 1/Infections:

The results of the infections are given in table 3. IP/ml: infectious particles per ml; ND: not determined.

TABLE 3

| Plasmids | No treatment | Rnase A | Dnase I | Actinomycin D | Geneticin |
|---|---|---|---|---|---|
| SFV 26Sm1 | 9 × 10³ IP/ml | 7 × 10³ IP/ml | 5 × 10³ IP/ml | 4.5 × 10³ IP/ml | <10² IP/ml |
| SFV 26Sm2 | 8 × 10³ IP/ml | 6 × 10³ IP/ml | 4.5 × 10³ IP/ml | 4.7 × 10³ IP/ml | <10² IP/ml |
| spSFV26Sm1 | 7 × 10³ IP/ml | 5 × 10³ IP/ml | ND | ND | ND |
| spSFV26Sm1 ψ | 6 × 10³ IP/ml | 4 × 10³ IP/ml | ND | ND | ND |

The presence of cells expressing GFP confirms the possibility of mobilizing SFV recombinant RNAs through the intervention of a retroviral particle. However, the low titres observed indicate that it is necessary to control the cytotoxicity of the SFV vector in order to obtain higher titres. This is because an antagonism exists between the production of the SFV RNAs and the production of the retroviral proteins. The production of the latter is decreased when the production of the SFV proteins increases. Several SFV mutants have, at this time, been described and may be beneficially used (8).

The presence or the absence of the retroviral encapsidation sequence does not seem to have a considerable influence on the encapsidation efficiency. Here, the high intracellular concentration of RNA appears to have a determining role in promoting the encapsidation, in agreement with the observations of Muriaux et al. (9). The influence of the psi retroviral sequence will have to be reevaluated in the context of low-toxicity vectors.

Moreover, these results appear to indicate that there is probably a contamination of recombinant retrovirus productions when a “helper” system based on SFV vectors is used (10, 11). These contaminants are made up of retroviral particles containing either the SFV vectors used to express the retroviral transcomplementation sequences, or the SFV vectors comprising the sequence of the recombinant retrovirus. This observation calls into question the use of these methods of production of retroviral vectors for clinical purposes, unlike the viral particles of the invention.

### BIBLIOGRAPHY

1. Rolls, M. M., Webster, P., Balba, N. H. & Rose, J. K. Novel infectious particles generated by expression of the vesicular stomatitis virus glycoprotein from a self-replicating RNA. Cell 79, 497-506. (1994).
2. Rolls, M. M., Haglund, K. & Rose, J. K. Expression of additional genes in a vector derived from a minimal RNA virus. Virology 218, 406-411. (1996).
3. Lebedeva, I., Fujita, K., Nihrane, A. & Silver, J. Infectious particles derived from Semliki Forest Virus Vectors encoding Murine Leukemia virus envelopes. Journal of Virology 71(9), 7061-7067. (1997).
4. Russell S J, Cosset F L. Modifying the host range properties of retroviral vectors. J Gene Med 1, 300-11. (1999).
5. Salonen A, Vasiljeva L, Merits A, Magden J, Jokitalo E, Kaariainen L. Properly folded non-structural polyprotein directs the semliki forest virus replication complex to the endosomal compartment. J Virol. 77, 1691-702. (2003).
6. Naldini, L. et al. In vivo gene delivery and stable transduction of non-dividing cells by a lentiviral vector. Science 272, 263-267 (1996).
7. Liu, M. L., Winther, B. L. & Kay, M. A. Pseudotransduction of hepatocytes by using concentrated pseudotyped vesicular stomatitis virus G glycoprotein (VSV-G)-Moloney murine leukemia virus-derived retrovirus vectors: comparison of VSV-G and amphotropic vectors for hepatic gene transfer. J. Virol 70, 2497-2502. (1996).
8. Lundstrom K, Abenavoli A, Malgaroli A, Ehrengruber MU. Novel semliki forest virus vectors with reduced cytotoxicity and temperature sensitivity for long-term enhancement of transgene expression. Mol Ther. 2, 7209-9. (2003).
9. Muriaux, D., J. Mirro, et al. RNA is a structural element in retrovirus particles. Proc Natl Acad Sci USA 98, 5246-51. (2001).
10. Wahlfors J J, Xanthopoulos K G, Morgan R A. Semliki Forest virus-mediated production of retroviral vector RNA in retroviral packaging cells. Hum Gene Ther 8, 2031-41. (1997)
11. Li K J, Garoff H. Packaging of intron-containing genes into retrovirus vectors by alphavirus vectors. Proc Natl Acad Sci USA 95, 3650-4. (1998).

1. Viral particle consisting of structural elements not derived from an alphavirus and containing an alphavirus-derived vector made replication-defective by deletion, or replacement with at least one transgene, of the structural genes, characterized in that the structural elements of said particle are not encoded by the genome of the alphavirus-derived vector.

2. Viral particle according to claim 1, characterized in that the structural elements correspond to the VSV-G envelope protein alone.

3. Viral particle according to claim 1, characterized in that the structural elements correspond to the structural proteins of a retrovirus.

4. Particle according claim 1, characterized in that the alphavirus is a Semliki forest virus.

5. Particle according to claim 1, characterized in that the genome of the alphavirus-derived vector contains the extended packaging sequence of MLV vectors.

6. Particle according to claim 1, characterized in that the genome of the alphavirus-derived vector is devoid of psi sequence.

7. Particle according to claim 1, characterized in that the genome of the alphavirus-derived vector comprises a 5′-positioned eukaryotic promoter.

8. Particle according to claim 1, characterized in that the alphavirus-derived vector contains a mutated p26S promoter.

9. Use of the viral particle of claim 1, for infecting a eukaryotic cell in vitro.

10. Pharmaceutical composition comprising the viral particle of claim 1.

11. Use of the viral particle of claim 1, for producing a medicinal product for use in the treatment of cancer.

12. Method for obtaining viral particles consisting of structural elements not derived from an alphavirus and containing an alphavirus-derived vector made replication-defective by deletion, or replacement with at least one transgene, of the structural genes, consisting:
in expressing in trans, in a cell line, the genes encoding the structural elements not derived from the alphavirus and the alphavirus-derived vector,
in recovering the viral particles present in the cell culture supernatant.

13. Method according to claim 12, characterized in that the structural elements correspond to the VSV-G envelope protein.

14. Method according to claim 13, characterized in that the expression in trans is obtained by cotransfection of a cell line with the vector for expressing the VSV-G envelope and the alphavirus-derived vector, the cotransfection being carried out in two distinct steps, respectively the transfection of the line with the vector expressing the VSV-G envelope gene, and then a second transfection with the alphavirus-derived vector.

15. Method according to claim 14, characterized in that the transfected cell line is a 293T cell line.

16. Method according to claim 12, characterized in that the structural elements correspond to the structural proteins of a retrovirus.

17. Method according to claim 16, characterized in that the expression in trans is obtained by transfection of an encapsidation cell line, that produces replication-defective retroviruses, with the alphavirus-derived vector.

18. Method according to claim 17, characterized in that the encapsidation cell line is obtained by stable transfection of a cell line with a first viral element expressing the retroviral GAG and POL genes and a second viral element expressing the retroviral ENV gene.

19. Method according to claim 16, characterized in that the expression in trans is obtained by triple transfection of a 293T cell line by introduction of a first viral element expressing the retroviral GAG and POL genes, of a second viral element expressing the retroviral ENV gene and of the alphavirus-derived vector.

20. Method according to claim 12, characterized in that the alphavirus is a Semliki forest virus.

21. Method according to claim 12, characterized in that the genome of the alphavirus-derived vector contains the extended packaging sequence of MLV vectors.

22. Method according to claim 12, characterized in that the genome of the alphavirus-derived vector is devoid of psi sequence.

23. Method according to claim 12, characterized in that the genome of the alphavirus-derived vector comprises a 5′-positioned eukaryotic promoter.

24. Method according to claim 12, characterized in that the alphavirus-derived vector contains a mutated p26S promoter.
